(19) United States
(12) Patent Application Publication
(10) Pub. No.: US 2007/0186935 A1
(43) Pub. Date: Aug. 16, 2007

(54) Coition activated inflatable reservoir condom

(76) Inventors: Francis Wang, Tustin, CA (US); Michael S. Shaw, Tustin, CA (US)

Correspondence Address: Francis Wang, 2216 Bowman, Tustin, CA, 92782-1036, US

(21) Appl. No.: 11/353,845

(22) Filed: Feb. 14, 2006

(51) Int. Cl. A61F 6/04

(52) U.S. Cl. 128/844

(57) ABSTRACT

A coition activated inflatable reservoir condom. The condom comprises a various number of inflatable tubular portions on its shaft and an anterior inflatable dome. A fluid filled reservoir attached to the base of the condom to supply the fluid into the inflatable portions by body pressure between the users. One handheld pump with a tube connected to the reservoir as the source of additional fluid for the reservoir. One receiving empty container with a relief valve also is included to release the fluid from the inflated portions and to reduce the size of the condom during the intercourse per users' discretion. This invention is a condom that provides for an enhanced sexual experience.

### FIELD OF THE INVENTION

The present invention is generally related to innovative and size enlargeable condom devices, more particularly, the present invention is related to a condom attached with a fluid filled reservoir which could be activated by users during the intercourse. The users are able to manipulate the fluid for controlling the size of the condom and for increasing stimulation and pleasure.

### BACKGROUND OF THE INVENTION

The change of the size of a condom by adding layers of materials or by injecting air into the air chambers to inflate the condom have been known in the prior arts. The enlargeable or inflatable condoms utilized for this purpose function on the principle that tubing or inflatable portions are attached to the condom. The size of the condom could be either predetermined or adjustable per user's discretion. Such devices are described as follows.

M. Maurice Rogers in U.S. Pat. No. 4,281,648 issued on Aug. 4, 1981, entire contents of which are incorporated herein by reference, discloses such an inflatable anterior portion and a pressure bulb combination. An air duct in communication with the anterior portion with the pressure bulb for manipulation of the inflatable condom.

Odysseus Tsagarakis in U.S. Pat. No. 5,823,939 issued on Oct. 20, 1998, entire contents of which are incorporated herein by reference, discloses a condom that is capable of expanding and contracting both circumferentially and longitudinally by two separate tubes. Two hand held pumps and connecting tubes are included to give users the option to change the desirable sizes during the sexual relations.

James D. Nordheim; Cathleen Nordheim in U.S. Pat. No. 6,193,753 B1 issued on Feb. 27, 2001, entire contents of which are incorporated herein by reference, discloses an external penile prosthesis comprises a support including an expandable bladder having first and second elastic layers. Once inflated, the fully expanded condition has supportive rigidity and adds substantially to the circumference of the penis.

Leon B. Kassman in U.S. Pat. No. 6,569,083 B1 issued on May 27, 2003, entire contents of which are incorporated herein by reference, discloses male and female condoms that exert lateral pressure on the shaft of the penis and the wall of the vagina respectively. An inflatable compartment and microtubules are provided for users to activate the lateral pressure within safe limits.

Brian J. Osterberg in U.S. Pat. No. 6,651,667 B2 issued on Nov. 25, 2003, entire contents of which are incorporated herein by reference, discloses a male condom with a flexible tubular body and a close end that conforms to the head of the penis, this anatomically configured condom does not distort the penile shape and allows for the normal action and sensations during the sexual intercourse. Further, the pockets with filler material are added along the shaft of the condom to enhance the shape and size of the user's penis.

Scott Praml in U.S. Pat. No. 6,895,967 B2 issued on May 24, 2005, entire contents of which are incorporated herein by reference, discloses a condom with an inner wall and outer wall defining an annular chamber. The chamber is connected with an internal airway and a removable handheld pump as the air source. The air travels along the internal airway into the air chamber, and allowing it to be inflated at desirable time. Air vents disposed at the base of the chamber allow for quick deflation. The handheld pump and associated flexible tube can be disconnected from the condom for reuse.

None of the devise is capable of expanding both circumferentially and longitudinally by a reservoir automatically squeezed between the users' body pressure to provide the option for controlling its expansion and to reach the desired size. This invention allows users to reach maximum comfort and pleasure during the sexual relations.

### SUMMARY OF THE INVENTION

The present invention related to a condom with a fluid filled reservoir attached to the open end. A prophylactic or prosthetic device with the conventional function to prevent unwanted pregnancy and protect against communicable diseases, as well as provide controllable enlargement portions to increase sexual pleasure for both male and female users. The condom comprises a various number of inflatable tubular portions along the shaft of its length, and an inflatable dome shape chamber at the anterior portion. A reservoir filled with fluid is attached to the top portion of the condom base to supply the fluid to the inflatable portions. The fluid inside the reservoir will be automatically squeezed into the condom during the intercourse when body pressure at the pubic areas between the partners pushed against each partner.

The condom further includes one handheld feeding pump with a tube connected to the reservoir. The pump is functioning as the source of additional fluid for the reservoir. The fluid filled feeding pump could be manually activated by the user and injects additional fluid into the reservoir to further enlarge the condom. One receiving empty container with a relief valve is provided to release the fluid from the inflated portions per user's discretion to reduce the size of the condom anytime during the intercourse. The reservoir attachment along with its associated devices may apply to both male and female condoms according to its design.

In alternative embodiments, the condom includes a circular donut shape reservoir or an U shape reservoir that attached to the base or the open end of the condom along with several compartments inside for feeding a pre-determine amount of fluid to the inflatable portions. The reservoir is to be squeezed between the base of the penis and the vulva during the intercourse. The reservoir gradually injects the fluid into the inflatable portion of the condom through the connecting microtubules. Further, a sodium acetate heat pad is added inside the reservoir to heat the fluid when triggered, and to enhance the sexual enjoyment.

### BRIEF DESCRIPTION OF THE DRAWINGS

To facilitate a clear understanding of the invention some particular embodiments will now be described with reference to the accompanying Drawings, in which:

FIG. 1 is a diagrammatic side elevation view of the present invention.

FIG. 2 is an alternative embodiment side elevation view of the present invention

FIG. 3 is a diagrammatic cross sectional view taken along section line D-D

FIG. 4 is a side elevation view of the present invention with a handheld pump and a storage container.

FIG. 5 is a plan view of the present invention with a handheld pump and a storage container.

FIG. 6 is a bottom view of the present invention with a handheld pump and a storage container.

FIG. 7 is a diagrammatic cross sectional view taken along section line A-A

FIG. 8 is a diagrammatic cross sectional view taken along section line B-B

FIG. 9 is a diagrammatic cross sectional view taken along section line C-C

FIG. 10 is a diagrammatic cross sectional view taken along section line E-E

FIG. 11 is a folded condom top view with two interior ears shown.

### DETAILED DESCRIPTION OF PREFERRED EMBODIMENTS

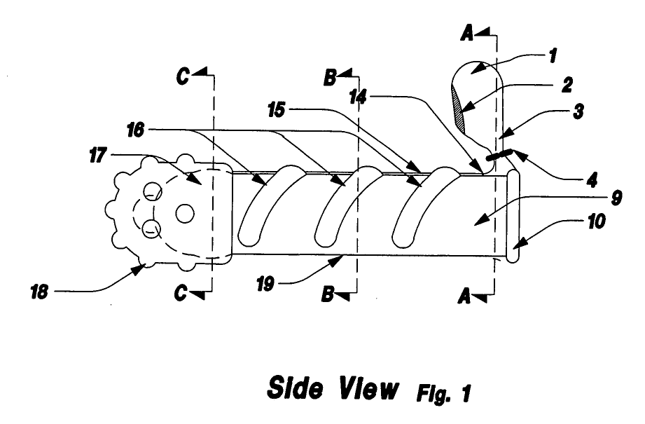

The preferred embodiments of the present invention described below relate to a male condom illustrated in FIG. 1 is formed of latex or polyurethane. The condom has an open end 10 with a ring, a closed anterior chamber 17 and a tubular wall 9 interconnecting both ends and is attached with inflatable tubular portions 16 along the shaft of the condom. The inflatable portions are used to increase the circumferential and the longitudinal size of the condom when fluid is injected into them. A reservoir 1 is attached to the top of the open end or the base of the condom above the condom ring 10, and there are several compartments 3 inside the reservoir 1 for pre-determined amount of fluid to be stored. Microtubules 15 connect the compartments to the designated inflatable portions of the condom. Furthermore, a sodium acetate heat pad 2 is attached inside the reservoir to heat the fluid when triggered.

An inflatable dome shape chamber 17 at the front end of the condom receives fluid from reservoir 1, once inflated, a substantially dome-shaped portion to be formed at the closed end, there are inflatable semispherical bumps 18 cover the dome to increase the sensation for female partner.

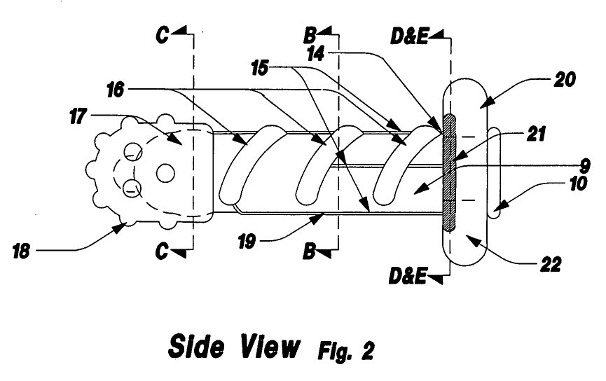

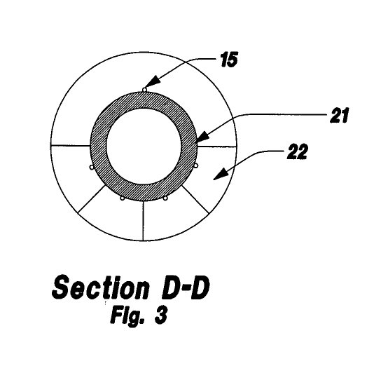

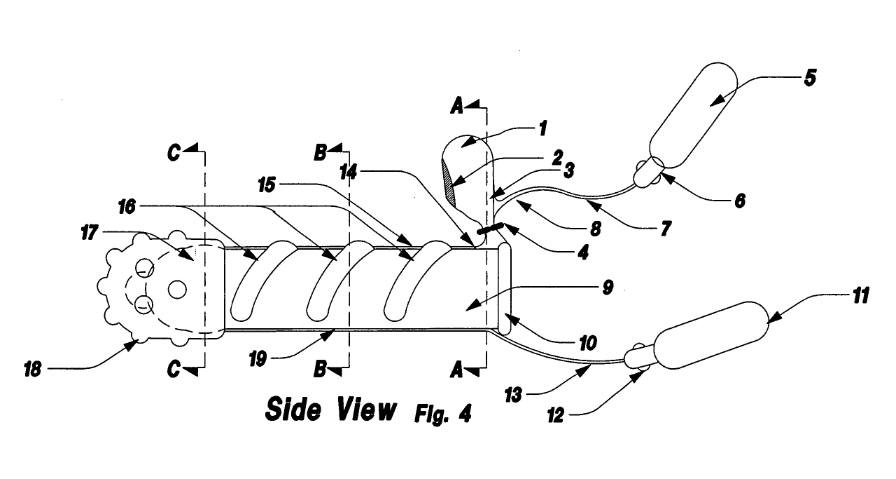

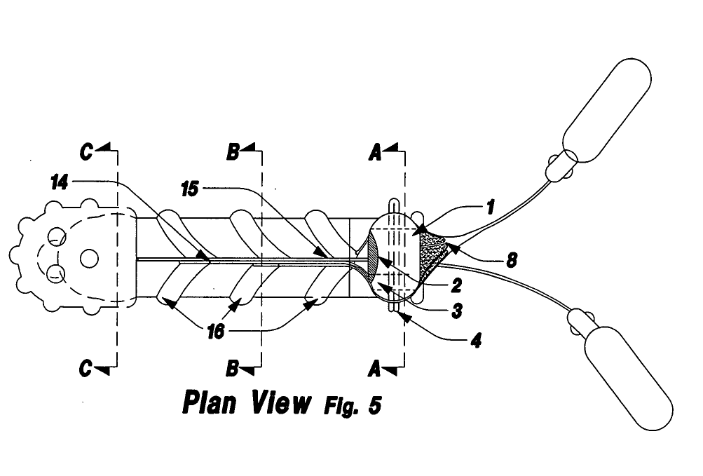

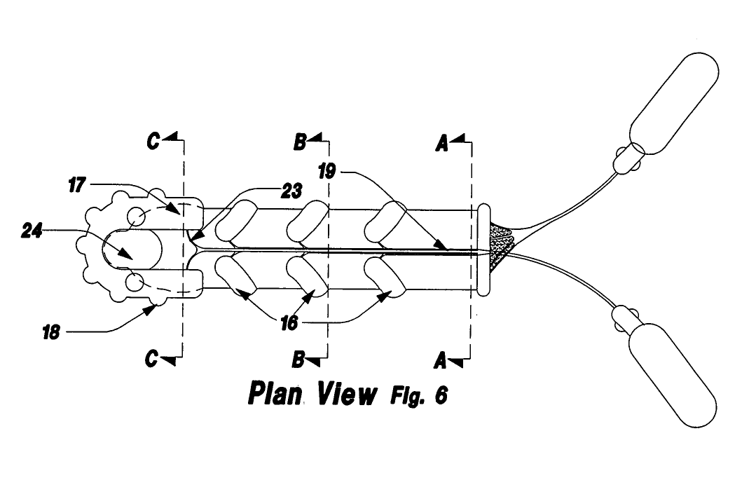

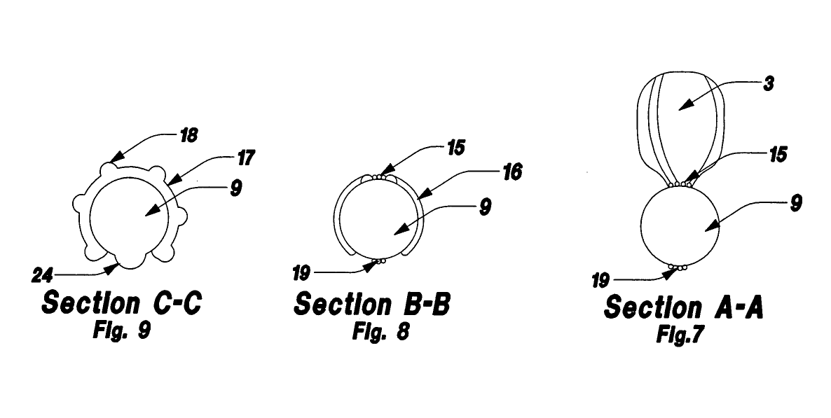

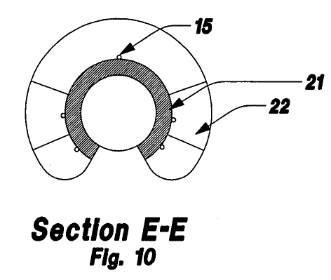

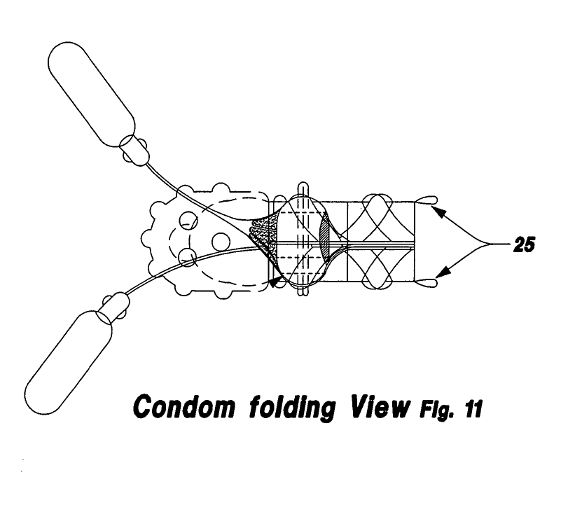

In alternative embodiments shown on FIG. 2, the condom includes a circular donut shape reservoir 20 & FIG. 3 or an upside down U shape reservoir 20 & FIG. 10 that attached to the base or the open end of the condom along with several compartments 22 inside for feeding a pre-determined amount of fluid to the inflatable portions, a circular sodium acetate heat pad 21 is included inside the reservoir. The reservoir is to be squeezed between the base of the penis and the vulva during the intercourse. While under body pressure, it gradually injects the heated fluid into the inflatable portions of the condom through the connecting microtubules 15.

A fluid filled handheld pump 5 with a control valve 6 are provided for adding fluid into the reservoir if needed. The handheld pump is connected with a tube 7 to the reservoir, and branches into microtubules 8 to distribute the fluid into the compartments in the reservoir. The diameters of the microtubules determine the amount of the fluid to be channeled into certain compartment.

A relief microtubules 19 and control valve 12 is provided to drain the excess liquid to the empty container 11 through relief tube 13 if needed in order to control the size of the inflatable portions.

The condom is packaged at a folding position FIG. 10, there are two ears 25 made of the same elastic material as the condom and attached to the interior wall at the middle section of the condom shaft. The ears to be held by both hands to help slip the condom onto the penis, after place half of the front end on the penis, and then the rest folded portion could be rolled over to complete the condom installation.

The condom should place over the penis and insert into the vagina of the female, prior to apply any of it functions. A control valve 6 is provided to stop the handheld pump from prematurely feed the liquid into the reservoir, and a clamp 4 is provided at the base of the reservoir to stop the fluid flow prior to the application of the condom. Once the condom is donned the penis, and after inserted into the vagina, the clamp 4 could be removed; the handheld pump control valve 6 could be click to open if needed.

The reservoir on FIG. 1 is designed to situate between the two partner's pubic area, it will automatically feed the fluid into the inflatable portions in the condom during the intercourse when it is squeezed between the partners at the pubic areas. The sodium acetate heat pad 2 could be triggered to lease the heat for the fluid inside the reservoir prior to the intercourse. A handheld pump 5 is connected with microtubules 8 to the reservoir; a single tube 7 is branched into a few microtubules 8 and feed the fluid into designated compartment 3 in the reservoir. The compartments 3 in the reservoir 1 store pre-determinated amount of fluid to each designated inflatable portion on the condom. The fluid filled handheld pump is able to continuously supply the reservoir with additional fluid and to be activated by the user if needed. The control valve 6 is also functioned to prevent backflow.

The handheld pump 5 is used to control the additional fluid for the reservoir, and the empty container 11 and the relief valve 12 is for reducing the size of the inflatable portions if needed during the coitus. By activating the relief valve 12, the fluid will drain away into the empty container through the relief tubules 19 & 23, which are connected from each inflatable portion to the container. There are also one way valves 14 inside each microtubule or at the inlet of the inflatable portions to prevent back flow to the reservoir while under pressure.

A close end dome shape (Section C-C) is specially designed to form a shallow pocket 24 at the bottom of the front end, so the space will not be squeezed out after the inflation, and is used for the storage of semen.

The condoms embodying the invention provide more fun and pleasure to use than those currently available. It will be understood that each of the elements described above, or two or more together, may also find a useful application in other types of constructions differing from the types described above.

While the invention has been illustrated and described as a reservoir attached, heat pad attached, inflatable and controllable condom for increasing the pleasure for the users, however, it is not limited to the details shown, since it will be understood that various omissions, modifications, substitutions and changes in the forms and details of the device illustrated and its operation can be made by those skilled in the art without departing in any way from the spirit of the present invention.

What is claimed is:

1. A fluid filled reservoir attached to the top portion on the open end of the condom, the reservoir connects with the inflatable portions of the condom with microtubules. The reservoir is to provide fluid for the inflatable portions of the condom.

2. A condom according to claim 1, wherein a sodium acetate heat pad from the prior art is placed inside the reservoir to heat the fluid inside the reservoir when the pad is triggered during the intercourse.

3. A fluid filled circular donut shape reservoir at the base of the condom and circles the open end of the condom. The reservoir is to provide fluid for the inflatable portions of the condom, when it is squeezed between the base of the penis and the vulva during the intercourse.

4. A condom according to claim 2, wherein a circular sodium acetate heat pad from the prior art is placed inside the donut shape reservoir to heat the fluid inside the reservoir when the pad is triggered during the intercourse.

5. A fluid filled upside down U shape reservoir at the base of the condom. The reservoir is to provide fluid for the inflatable portions of the condom, when it is squeezed between the base of the penis and the vulva during the intercourse.

6. A condom according to claim 5, wherein an upside down U shape sodium acetate heat pad from the prior art is placed inside the U shape reservoir to heat the fluid inside the reservoir when the pad is triggered during the intercourse.

7. A condom according to claim 1, 3 & 5, wherein several compartments are provided inside the reservoir in order that pre-determined amount of fluid to be channeled into its designated inflatable portions of the condom through the attached microtubules. The size of the compartment in the reservoir is used to control the amount of fluid flows into the inflatable portion.

8. A condom according to claim 1, wherein the reservoir is designed to situate between the two partners' pubic areas, it will automatically and gradually feed the fluid into the inflatable portions in the condom during the intercourse when it is squeezed between the partners at the pubic areas.

9. A condom according to claim 3 & 5, wherein the reservoir is designed to situate between the base of the penis and the vulva. It will automatically and gradually feed the fluid into the inflatable portions in the condom during the intercourse when it is squeezed between the base of the penis and the vulva.

10. A condom according to the claim 1, wherein a handheld pump filled with fluid is provided and connected to the reservoir by a tube for allowing manually adding additional fluid into the different compartments in the reservoir. The handheld pump supplies the fluid to the reservoir; the reservoir in term continuously feeds the inflatable portions of the condom to further enlarge the condom.

11. A condom according to the claim 10, wherein the tube from the handheld pump is branched into a few microtubules for feeding the certain amount of fluid into the designated compartments in the reservoir. The size of the microtubule is to determine the amount of fluid to be feed into the reservoir.

12. A condom according to claim 1,3 & 5, wherein all the microtubules along the length of the condom is provided with one way valves to prevent backflow.

13. A condom according to claim 1, 3 & 5, wherein an inflatable fluid stored dome-shape portion attached with fluid inflatable semispherical bumps at the end of the condom to increase sensation for female user are provided.

14. A condom according to claim 1, 3 & 5, wherein inside the inflatable dome-shape front end of the condom a pocket is provided at the crop out bottom portion of the dome-shape for the storage of the ejected semen.

15. A condom according to claim 10, wherein relief microtubules connect to each of the inflatable portion, and a manually release valve is provided to drain away the fluid inside the inflatable portions of the condom per users' discretion.

16. A condom according to claim 15, wherein an empty container is attached at the end of the relief microtubules to store the released fluid.

17. A condom according to claim 1, wherein a removable clamp is provided at the base of the reservoir to block the fluid from entering into the inflatable portions of the condom prior to the intercourse. The clamp is removed after the condom is placed over the penis, and before inserting into the vagina, to allow the fluid flowing from the reservoir to the inflatable portions.

18. A condom according to claim 1, wherein the condom could be in a folding position in the package. Two ears made of same elastic material of the condom are provided at the mid-point of the condom interior wall. These will be used to help the initial application of the condom and to slip the condom onto the penis, after that, the rest of the condom could be rolled over to complete the installation on the penis.
